(19) United States
(12) Patent Application Publication
(10) Pub. No.: US 2014/0243737 A1
(43) Pub. Date: Aug. 28, 2014

(54) ASSAYS FOR ULTRASOUND MEDIATED DELIVERY

(71) Applicant: KONINKLIJKE PHILIPS N.V., EINDHOVEN (NL)

(72) Inventors: Todd Nicholas Erpelding, University city, MO (US); Ralf Seip, Carmel, NY (US); Christopher Stephen Hall, Kirkland, WA (US); Balasundar Iyyavu Raju, Chester, NY (US)

(21) Appl. No.: 14/349,496

(22) PCT Filed: Sep. 25, 2012
(86) PCT No.: PCT/IB2012/055087

§ 371 Date: Apr. 3, 2014

Related U.S. Application Data
(60) Provisional application No. 61/543,469, filed on Oct. 5, 2011.

(51) Int. Cl. A61M 37/00

(52) U.S. Cl. CPC A61M 37/0092
USPC 604/22

(57) ABSTRACT

Ultrasound mediated delivery (USMD), real-time quantitative feedback derived (S264) therefrom, and proceeding by the system based on the feedback all are, in some embodiments, operable automatically and without need for user intervention. USMD may occur in a clinical setting accompanied by assays (S276) or real-time feedback, or by means of a wearable device that, based on feedback, regulates USMD in real time. Optionally, the user is provided an indication (S281) as to progress or success, of a treatment. Electrodes (128) may be attached across tissue in which transient pores are produced via sonoporation in the USMD procedure, and in vivo measurement is taken of an electrical parameter responsive to permeability. Therapeutic agent (S202) may be administered after particles activated for sonoporation are cleared from the circulation, to avoid, when it might exist, adverse interaction between the particles and agent.

### FIELD OF THE INVENTION

The present invention relates to ultrasound mediated delivery and, more particularly, to quantitative feedback and proceeding based on the feedback.

### BACKGROUND OF THE INVENTION

Ultrasound meditated delivery (USMD) of drugs, genetic materials, and other therapeutic agents are promising applications of ultrasound therapy. In these approaches, particles (nanoparticles, liposomes, microcapsules, microbubbles, etc.) incorporate therapeutic agents onto the surface, within the outer coating, within the core of the particle, or in proximity of the particles. Spatially localized treatments are achieved by site-targeted delivery with specific targeting ligands, but also through exposure of a volume of tissue to activating ultrasound energy. Targeting ligands enable binding to specific pathological epitopes and can be incorporated onto the particle surface through avidin-biotin linkages, chemical, or electrostatic interactions. Ultrasound is then introduced to enhance release of the drug.

The mechanisms for ultrasound mediated delivery are dependent on the type of particle and ultrasound exposure, but can be generally characterized as mechanical (pressure, radiation force, acoustic cavitation) or thermal effects. The mechanical effects can be referred to as “pressure-mediated effects” and the thermal effects can be referred to as “temperature-mediated effects.” As to the latter, commonly-assigned International Patent Publication WO 2010/029469 to Langereis et al., entitled “Drug Carrier Providing MRI Contrast Enhancement” is directed to the USMD delivery by melting liposomes so that MRI contrast agent therein is then active, and to control of local drug delivery under MRI-imaging guidance.

U.S. Patent Publication No. 2008/0200838 to Goldberger et al. (hereinafter “the ‘838 application’”), entitled “Wearable, Programmable Automated Blood Testing System”, discloses a device that both samples blood and tests the samples automatically. Wearable devices that deliver ultrasound therapy are disclosed in U.S. Patent Publication No. 2011/0066083 to Tosaya et al. (hereinafter “the ‘083 application’”), entitled “Ultrasonic Apparatus and Method for Treating Obesity or Fat-Deposits Or For Delivering Cosmetic Or Other Bodily Therapy”, and in U.S. Patent Publication No. 2010/0130891 to Taggart et al. (hereinafter “the ‘891 application’”), entitled “Wearable Therapeutic Ultrasound Article.” U.S. Patent Publication No. 2004/0236375 to Redding (hereinafter “the ‘375 publication’”), entitled “Wearable, Portable Sonic Applicator For Inducing The Release Of Bioactive Compounds From Internal Organs”, discloses applying ultrasound directly to the pancreas to cause release of insulin. U.S. Pat. No. 7,763,582 to Lin et al. (hereinafter “the ‘582 patent’”), entilted “Localized Insulin Delivery for Bone Healing”, is directed to a surgically-implantable drug delivery device for delivering insulin, and to using liposomes for delivering insulin. The entire disclosure of the above documents is incorporated herein by reference. None of the above mentioned documents discloses activating particles to deliver, as a payload, a therapeutic agent.

### SUMMARY OF THE INVENTION

Current development of ultrasonic techniques to enhance delivery of drugs, genetic materials, or other therapeutic agents is hindered by a lack of methods to monitor treatments. No such quantitative treatment feedback currently exists for mechanical (pressure, radiation force, acoustic cavitation) USMD treatments, making treatment monitoring, final dosage assessments, and, ultimately, treatment success estimation difficult. Overall, further development is desired of USMD monitoring of mechanical and thermal effects to help guide research efforts and accelerate clinical translation.

Disclosed herein below are several devices and methods capable of providing quantitative treatment feedback for ultrasound mediated delivery treatments taking the form of blood assays, tracer protein expressions, and/or targeted imaging techniques. Occurring before, during, and/or after the ultrasound mediated delivery treatment, these methods inform the clinician as to whether the desired ultrasonic and therapeutic agent dosage was, in fact, delivered to the targeted tissue. If not, adjustments in the treatment plan and execution can be made to achieve the desired dosage level prior to treatment completion and patient discharge. In addition, the feedback is, where appropriate, derived, and analyzed, in real time for automated decisions on continuing or repeating treatment.

In an aspect of the present invention, a device inclues an ultrasound therapy module configured for, automatically and without need for user intervention, performing ultrasound mediated delivery so as to deliver a therapeutic agent via pressure-mediated, rather than temperature-mediated, effects. The device is configured for, automatically and without need for user intervention, proceeding based on quantitative feedback derived from the delivering.

In a sub-aspect, the proceeding includes at least one of: a) providing a user indication as to at least one of progress, and success, of a treatment; and b) deciding whether to continue or repeat a treatment.

In another sub-aspect, the device further includes an imaging module, and a processor configured for the deriving in real time, automatically and without need for user intervention. The deriving relies on real-time imaging by the imaging module.

In a further sub-aspect, an energy beam is issued for, through the above-mentioned effects, destroying particles in performing the delivery. The imaging includes imaging a result of the destruction.

In another sub-aspect, the imaging is of a modality other than ultrasound.

In one further sub-aspect, an energy beam, of other than said modality, is issued to afford the deriving by activating a contrast agent of said modality.

In a yet further sub-aspect, the beam carries a radiofrequency pulse sequence specific for the activating.

In an alternative sub-aspect, the modality is fluorescent based or photoacoustically based.

In a different aspect, the imaging is performed for detecting an amount of tracer substance released in the delivery. The feedback is based on the detected amount.

In a sub-aspect, the agent and tracer substance are administered, and the deriving includes determining, based on the detected amount, an acoustic parameter to maximize release, in real time, of the tracer substance.

In a related sub-aspect, electrodes are attachable to, following production of temporary pores, derive real-time quantitative feedback for ultrasound mediated delivery, the delivering and deriving both being performed automatically and without the need for user intervention. The deriving includes taking in vivo measurement, across the membranes, of an electrical parameter indicative of permeability.

In a further sub-aspect of the above, ultrasound is applied and/or bubbles are injected, in proceeding automatically and without the need for user intervention, and responsively in real time to the feedback comprising the electrical parameter.

According to a version, ultrasound mediated delivery is repeated or continued based on quantitative feedback that is based on pressure-mediated, rather than temperature-mediated, effects.

The feedback may be derived from in vitro diagnostic tests performed, respectively, before and after, said delivery delivers a therapeutic agent.

At least one of the tests may be a: a) blood-based assay, b) urine-based assay, and/or c) to measure molecular expression, a test utilizing a bodily substance other than blood or urine.

In another version, a wearable device includes an ultrasound mediated delivery module for applying ultrasound to particles to deliver, as a payload, a therapeutic agent. It further includes a unit configured for, automatically and without need for user intervention, both sampling body fluid and analyzing the samples.

In a sub-version, the device is configured for, automatically and without need for user intervention, regulating the delivering responsive to a result of the analysis.

In a different aspect, in either order for an ultrasound mediated delivery method for which quantitative feedback is derived, a) a transient pore through which, by the delivery, a therapeutic agent is to be delivered may be produced; and b) electrodes may be attached, to, following the producing and in performing the deriving, take in vivo measurement of an electrical parameter responsive to permeability.

Alternatively or in addition, for a device configured for ultrasound mediated delivery, energy may be applied to produce a transient pore through which to perform the delivery. After the pore is produced, the agent may be administered for said delivery through the pore.

Optionally, the administering may be deferred until after a site to which the applying is directed is clear of injected bubbles.

Details of the novel, closed-loop USMD device and methods are set forth further below, with the aid of the following drawings, which are not drawn to scale.

### BRIEF DESCRIPTION OF THE DRAWINGS

FIG. 1 is a schematic diagram of an automatic, quantitative-feedback-based USMD system;

FIGS. 2A, 2B, 2C are flow charts of a USMD procedures based on quantitative feedback; and

FIG. 3 is a schematic diagram of a wearable, self-regulating USMD device.

### DETAILED DESCRIPTION OF EMBODIMENTS

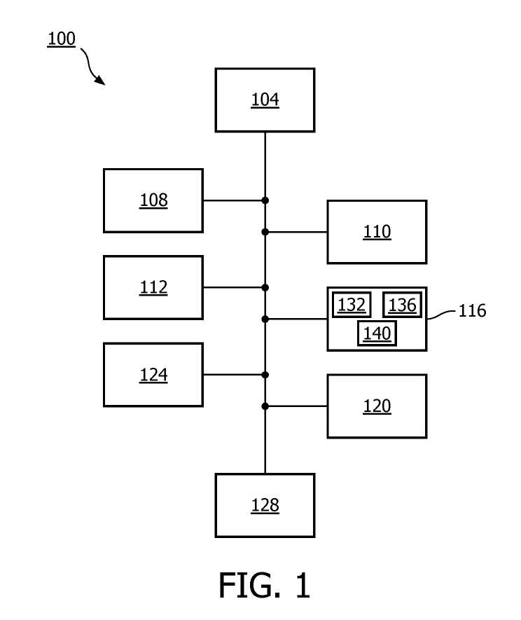

An automatic, quantitative-feedback-based USMD system 100, as shown in one possible configuration in FIG. 1, includes a control module 104, a user input module 108, a display module 110, an ultrasound therapy module 112, an imaging module 116, an injection module 120, a processor 124 and electrodes 128. The imaging module 116 includes one or more of the following: an ultrasound imaging unit 132, a magnetic resonance imaging (MRI) unit 136, and an optical unit 140 for fluorescent-based and/or photoacoustically-based imaging.

A device, as claimed herein below, may be implemented as the system 100, the control module 104, or one or more integrated circuits embodying an algorithm for quantitative-feedback-based USMD. The algorithm can reside in any kind of read-only memory (ROM) or random access memory (RAM), and may be received by the controller 104 by wire input, or wirelessly via an antenna and from a remote transmitting antenna. In either case, the signal to be transmitted is generated by appropriately varying an electrical current.

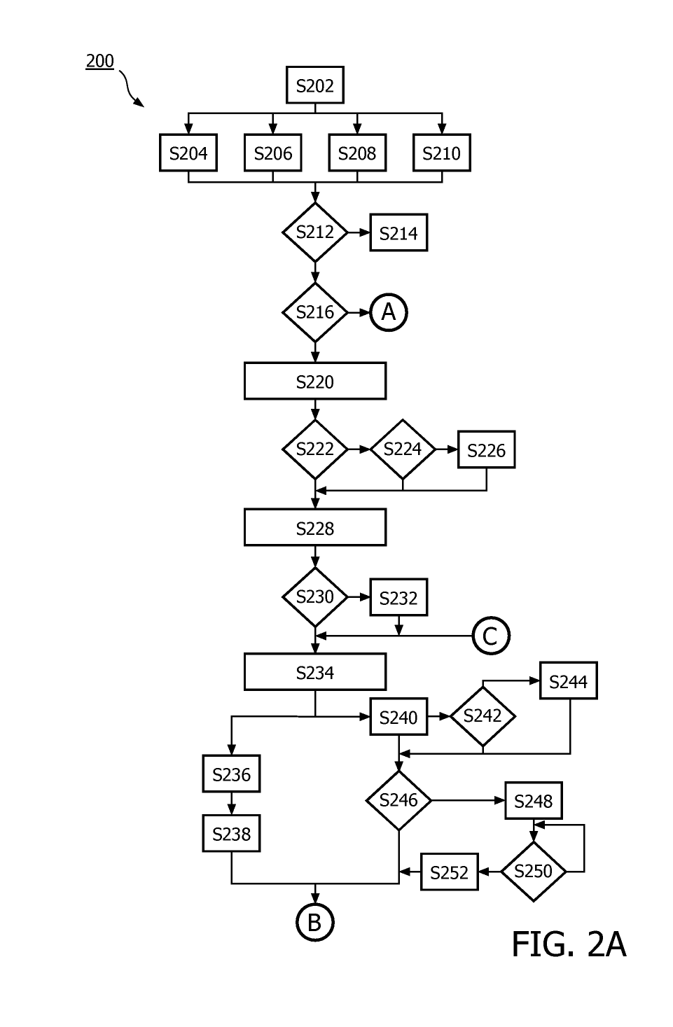

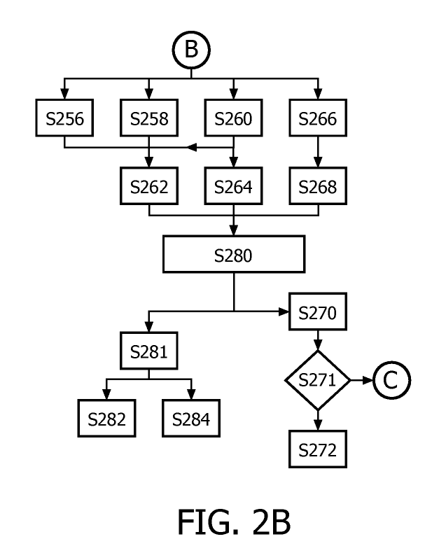

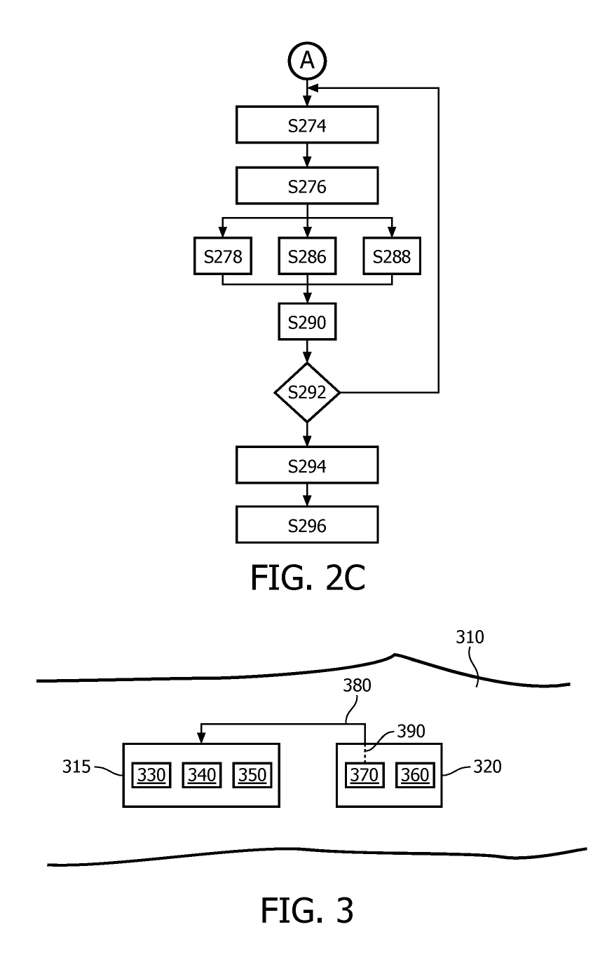

FIGS. 2A and 2B provide, by illustrative and non-limitative example, a quantitative-feedback-based USMD procedure 200. Initially, particles such as microbubbles; nanobubbles or other nanoparticles; liposomes; and microcapsules are configured with regard to size distribution and composition. A factor taken into account is the particular payload therapeutic substance, such as a drug or genetic material, that will be delivered under USMD (step S202). The particles may be configured with any one or more of drugs (step S204), genes (step S206), ligands, such as small peptides, antibodies, peptidomimetics, aptamers or other targeting molecules, conjugatable to the surface, within the outer coating, or with the core of the particle (step S208) and contrast agent (step S210) of the appropriate imaging modality.

Optionally, a baseline assay of the patient or animal subject may initially be taken for comparison with a subsequent assay to assess the results of USMD.

If the assays are to be taken on a periodic, ongoing basis, such as every two hours or every day, by a wearable device (step S212), then preparation proceeds for the wearable device.

The wearable device may be worn around the waist by means of supporting articles similar to those shown in the '375, '803 and '891 applications, for example. In place of the transducer of the '891 application, an imaging transducer can be confocally arranged within a therapy transducer. The device could further include the lancet and the blood or plasma analyte measuring element pairs of the '838 application. As in the '838 application, the device would derive feedback from glucose test strips or sensors. The device would, within or attached to the article, include an injection system for infusing particles configured in steps S202-S210. Thus, particles, such as liposomes or microbubbles, bearing insulin may be, under image guidance, ultrasonically activated. The activation, in a pressure- or temperature-mediated mode, releases the therapeutic payload of the particles so as to thereby promote bone healing in a non-diabetic patient, as in the '582 patent. Here, however, ultrasound is applied, to thereby deliver the therapeutic agent, and the device monitor in real time, automatically and without the need for user intervention. The monitoring is by means of the consequential take up of glucose by body tissue and resultant lowering of blood or plasma glucose level. Thus, for instance, if the level is lower than a pre-set threshold, insulin infusion is increased. Conversely, if the level is greater than or equal to the threshold, infusion is decreased. This process can be slow and continual over a span of days with body fluid testing perhaps every two hours. Targeted, local delivery, and its monitoring, are continual and automatic. Alternatively or in addition, the device, by means of a user interface such as that of the '375 application, can notify the user audibly and visibly for example, that an adjustment is needed to the ongoing therapy. At that point, the patient may be afforded action, limited by safety considerations, or can get medical assistance. There is an attachment port for cable connection to an ultrasound display device so that the imaging, which the device may use for self-regulating, can be utiltized by the clinician in initially orienting the transducer. As an alternative to the port, the connection is wireless. The self-regulating according to the imaging can include withholding application of ultrasound until a preset level of echogenecity representative of microbubbles, laden with the therapeutic agent, is within view. It also may include slowing or accelerating the infusion of microbubbles to achieve a targeted dosage rate.

In preparing the wearable device, a container with the pre-configured particles, bearing or otherwise in proximity of the therapeutic agent, will be attached to the injection system in the garment. By means of imaging, region of interest (ROI) is identified. The lancets, and an intravenous (IV) extension of the injection system, are engaged to the body of the patient or subject. The lancets, as described in the '838 application, can be contained in a single cartridge or cassette. The therapy and imaging transducers are, under image guidance, disposed so as to be directed to the ROI. An example of the therapy transducer would be an unfocused transducer.

If the wearable device will not be used, but assays not part of an automated feedback-based control loop are to be performed (step S216), then processing proceeds as shown in FIG. 2C. Otherwise, in preparation for an automatic feedback-based control loop, pre-treatment imaging is performed to identify the volume of interest, or “region of interest” (ROI) (step S220). If the USMD will involve sonoporation and if the sonoporation is to be monitored (step S222), query may be made as to whether electrodes 128 are to be attached at this time (step S224). If the electrodes 128 are to be attached now (step S224), a pair or configuration of several electrodes is attached to the patient/subject at the ROI (step S226).

In further preparation for treatment (step S228), the particles are loaded into the injection module 120. A package of the pre-configured particles may be placed into the module 120. The injection module 120 is connected to the patient/subject via a catheter, such as an intravenous (IV) line, or via a needle. Particle injection or infusion is commenced. Administration may thereafter be controlled either by the control module 104 or by the clinician. After the start of injection or infusion, the procedure 100 waits for a predetermined length of time for optimal ultrasound activation. Alternatively, the control module 104 monitors particle infusion using image guidance from the imaging module 116.

A specific radio frequency (RF) pulse sequence might be applied to activate an imaging contrast agent. MRI-based CEST (chemical exchange saturation transfer) and paraCEST agents, for example, whose imaging contrast is otherwise quenched or undetectable, can be selectively activated by such a sequence. The agents can be used in the measurement of pH, temperature, or concentration of metabolites following USMD.

If a specific RF pulse sequence is to be applied (step S230), the corresponding setting is made in the ultrasound therapy module 112 (step S232).

In either case, then an ultrasound beam issues as part of the USMD procedure (step S234).

As a result of the issuance, particles in the treatment site are activated (step S236). Any of the activated particles bearing or otherwise in proximity of the therapeutic agent thereby release their payload, if any, (step S238) for local uptake by the body tissue.

In addition, the intensity of the applied energy beam, here ultrasound, may, in conjunction with particle parameters, be such as to cause sonoporation (step S240) which creates transient holes, i.e., pores, in cell membranes through which the agent may enter the cell. If electrodes 128 are not already attached (step S242), they are now attached (step S244).

If the agent to be delivered is incompatible with the particles sonified (step S246) due to sensitivity of one to the other, the agent may be injected/infused afterward. In particular, the particles can be configured back in step S2020 so as to break up during sonoporation, and are subsequently cleared away by blood circulation within a minute or two. The pores persist for some time even after the broken particles are cleared away. After this time, the agent can be administered for intracellular take up at the treatment site. To localize release of the administered therapeutic agent, the agent may be borne by particles for thermally-activated delivery at the treatment site. Optionally, the release mechanism could be mechanical (i.e., ultrasound pressure-mediated). Accordingly, if the agent and particles are incompatible (step S246), infusion of microbubbles is halted (step S248). Once the treatment site is clear of the particle fragments (step S250), the agent is infused for take up, that optionally may be designed for assistance by local delivery activation, as described above (step S252).

Whether or not sonoporation has been induced, the feedback is now derived in accordance with FIG. 2B.

Alternatively, or in addition, deriving, by the processor 124, may draw on real-time imaging, or other real-time monitoring, of the results, discussed just above in steps S236-S240, of issuing the ultrasound beam in step S234.

The real-time imaging can include ultrasound imaging by the ultrasound imaging unit 132 (step S256).

It can also include optical imaging (step S258), such as fluorescent-based imaging or photoacoustic imaging, by the optical unit 140. For instance, some near-infrared fluorescent (NIRF) particles can act as contrast agents, but their imaging contrast is quenched or undetectable until specific excitation occurs, e.g., by enzymatic reactions. In particular, the NIRF particles are cleaved by specific proteases and release an optically-detectable fluorochrome. The enzymes or proteases needed for contrast agent activation could be released via ultrasound-triggered particles or produced via ultrasound-mediated gene transfection. Alternatively or in addition, photoacoustic imaging would be capable of reaching greater depths and better spatial resolution than fluorescence. Microbubbles or nanoparticles could be loaded with an optical dye that could be detected photoacoustically (absorption-based rather than fluorescent-based) to quantify drug delivery.

The real-time imaging might also include MRI (step S260) by the MRI unit 136, as noted above in connection with step S230. As another example, the injected particles may contain a protein, molecule, imaging-specific contrast agent, or other tracer substance released following ultrasonic activation. For instance, thermally sensitive liposomes can be configured in step S202 for releasing a Gd-based MRI contrast agent in response to ultrasound-induced heating, where the agent is not, by MRI-based contrast imaging, detectable in the bound state. The amount of released agent is indicative of the delivered therapeutic dose and useful for determining optimal acoustic parameters to maximize release. The amount of substance released is detected by the real-time imaging of the respective modality (step S262) and, as mentioned further above, for the MRI-based CEST and paraCEST agents, further observations with regard to pH and temperature may be acquired (step S264). It is within the intended scope of the invention that other imaging modalities such as positron emission tomography (PET), single-photon emission computed tomography (SPECT) or computed tomography (CT) might alternatively, or in addition, be employed. With respect to imaging contrast agents, the particles may incorporate contrast agents of various imaging modalities. These include microbubbles for ultrasound, Gd or FeO particles for MRI, radiolabeled particles for nuclear medicine, etc. Thus, immediate feedback is available that the particles have reached the target treatment region. Non-image-based monitoring includes monitoring for an electrical parameter (step S266). For example, cell membrane pore formation, as a result of sonoporation, in step S240, induces change in membrane resistance and conductivity. This change can be monitored in real time using the electrodes 128 attachable in, for example, steps 224 or 244. Based on a detected electrical parameter, the amount of substance intracellularly delivered can be estimated (step S268). The sonoporation that opens the pores may be supplemented by a subsequent infusion of particles such as liposomes for targeted delivery, through the still open pores, of a therapeutic payload via thermally-based USMD. Electrical impedance can also be monitored for changes in calcium uptake following ultrasound-mediated gene transfection.

For any of the monitoring modes, processing proceeds based on the derived feedback (step S280). The feedback comes, optionally in part, from a user indication (S281) on the display module 110 as to progress (step S282) or success (step S284) of the treatment. The indication of progress may entail a screen message on the current estimate of medicament dosage delivered. The indication of success may be a screen message that the target dosage has been delivered. Alternatively or additionally to the screen feedback, delivery activation is halted (step S270), but, here, the halting is done automatically by the control module 104. Processing then proceeds under the control and decisionmaking of the control module 104 and without the need for user intervention. In particular, if treatment is to continue or repeat (step S271), processing branches back to step S234. If treatment is not to continue or repeat (step S271), treatment is complete (S272)

An example of a processing path that can proceed automatically and without need for user intervention is S234, S236, S238, S256, S262, S280, S270, S271, S234. Many alternative paths likewise can proceed automatically and without the need for user intervention.

In FIG. 2C, once the baseline assay has been completed, ultrasound mediated delivery is performed (step S274), and a post-treatment assay is done (step S276).

The baseline assay and its complementary post-treatment assay may be based on blood (step S278), urine (step S286), or, to measure molecular expression, another bodily substance (step S288). The molecule whose expression is measured can be an enzyme or other proteins. Expressions of the selected protein or proteins are directly modulated by ultrasound-mediated drug/gene delivery. Changes from baseline values could indicate treatment success, or if additional treatments are required. An example is prostate specific antigen (PSA) levels in the blood as an indicator of prostate cancer (baseline measurement), its rapid rise 1-2 days after a thermal prostate cancer treatment due to the treatment itself, and its drop to below baseline, providing a quantitative measure of treatment efficacy.

Another example of an assay is one that involves ultrasound-mediated gene delivery, including plasmid DNA transfection or RNA interference (RNAi, siRNS, miRNA). After the delivery of the gene with ultrasound, the gene then up-regulates or down-regulates expression of a specific protein. Quantitative treatment efficacy feedback is provided by comparing pre- and post-treatment protein expressions. The post-treatment assay may be performed perhaps days later. An assay for change in calcium uptake, as an additional example, could indicate efficacy of ultrasound-mediated gene transfection.

As a further example, the particles could incorporate therapeutic agents and specific molecules, such as enzymes or other proteins, to block expression of cell membrane proteins or integrins. The amount of the agent delivered can be inferred by measuring changes in integrin expression.

The post-treatment assay is compared to the baseline assay (step S290).

If, based on a result of the comparison, it is decided that treatment is to continue or repeat (step S292), processing returns to step S274.

If, on the other hand, treatment is not to continue or repeat (step S292), the inflow of agent is halted (step S294), and treatment is terminated (step S296).

FIG. 3 shows, by way of example, a schematic diagram of a wearable, closed-loop USMD device or system 300. It includes, attached to belt 310 or other garment, a USMD module 315 and an analzying and sampling unit 320. The USMD module 315 includes an imaging transducer 330, a therapy transducer 340 and an injection system 350. The analyzing and sampling unit 320 includes a sampling sub-unit 360, and an analyzing unit 370 for analyzing the samples. The USMD module 315 regulates 380 the USMD responsive to a result 390 of the analysis.

Ultrasound mediated delivery (USMD), real-time quantitative feedback derived therefrom, and proceeding by the system based on the feedback all are, in some embodiments, operable automatically and without need for user intervention. USMD may occur in a clinical setting accompanied by assays or real-time feedback, or by means of a wearable device that, based on feedback, regulates USMD in real time. Optionally, the user is provided an indication as to progress or success, of a treatment. Electrodes may be attached across tissue in which transient pores are produced via sonoporation in the USMD procedure, and in vivo measurement is taken of an electrical parameter responsive to permeability. Therapeutic agent may be administered after particles activated for sonoporation are cleared from the circulation, to avoid, when it might exist, adverse interaction between the particles and agent.

Applications of ultrasound-mediated drug and gene delivery include oncology and chemotherapy, thrombolysis, treatment for cardiovascular diseases, and delivery across the blood-brain barrier. Some treatment feedback mechanisms employed for ultrasound-mediated delivery could be applicable to other ultrasound therapies, including high intensity focused ultrasound (HIFU) ablation, or drug development, in general.

While the invention has been illustrated and described in detail in the drawings and foregoing description, such illustration and description are to be considered illustrative or exemplary and not restrictive; the invention is not limited to the disclosed embodiments.

For example, although one therapeutic agent and associated delivery particles are described for administration, the injection module may be programmed for administration, automatically and without need for user intervention, of more than one agent and respective particles.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. Any reference signs in the claims should not be construed as limiting the scope.

A computer program can be stored momentarily, temporarily or for a longer period of time on a suitable computer-readable medium, such as an optical storage medium or a solid-state medium. Such a medium is non-transitory only in the sense of not being a transitory, propagating signal, but includes other forms of computer-readable media such as register memory, processor cache, RAM and other volatile memory.

A single processor or other unit may fulfill the functions of several items recited in the claims. The mere fact that certain measures are recited in mutually different dependent claims does not indicate that a combination of these measures cannot be used to advantage.

What is claimed is:

1. A device comprising:
an ultrasound therapy module (112) configured for, automatically and without need for user intervention, performing ultrasound mediated delivery so as to deliver a therapeutic agent via pressure-mediated, rather than temperature-mediated, effects, said device being configured for, automatically and without need for user intervention, proceeding based on quantitative feedback derived from the delivering.

2. The device of claim 1, said proceeding comprising at least one of: a) providing a user indication as to at least one of progress, and success, of a treatment (S281); and b) deciding whether to continue or repeat a treatment (S271).

3. The device of claim 1, further comprising an imaging module (116), and a processor (124) configured for the deriving in real time, automatically and without need for user intervention, said deriving relying on real-time imaging by said imaging module.

4. The device of claim 3, further configured for issuing an energy beam (S234) for, through said effects, activating particles in the performing of said delivery, said imaging being relied on comprising imaging of a result of the activation.

5. The device of claim 4, said proceeding comprising at least one of: a) providing a user indication as to at least one of progress, and success, of a treatment (S281); and b) deciding whether to continue or repeat a treatment (S271).

6. The device of claim 3, said imaging being of a modality other than ultrasound.

7. The device of claim 6, further configured for issuing an energy beam, of other (136) than said modality, to afford said deriving by activating a contrast agent of said modality (132).

8. The device of claim 7, said beam carrying a radiofrequency pulse sequence specific for the activating (S230).

9. The device of claim 7, said modality (140) being at least one of fluorescent-based and photoacoustically-based.

10. The device of claim 6, said imaging being performed for detecting an amount of tracer substance released in said delivery (S262), said feedback being based on the detected amount.

11. The device of claim 10, further comprising an injection module (120) for administering said agent and said substance, said deriving comprising determining, based on the detected amount, an acoustic parameter to maximize release, in real time, of said substance.

12. A device comprising:
an ultrasound therapy module configured for, automatically and without need for user intervention, performing ultrasound mediated delivery of a therapeutic agent;
a processor configured for, automatically and without need for user intervention, deriving, in real time, quantitative feedback; and
electrodes (128) attachable to, following production of transient pores, perform said deriving, said deriving comprising taking in vivo measurement, across said pores, of an electrical parameter indicative of permeability.

13. The device of claim 12, further comprising a control module configured for, automatically and without need for user intervention, in proceeding responsively in real time to said feedback comprising said parameter, at least one of applying ultrasound (S234) and injecting bubbles.

14. A method comprising:
based on quantitative feedback (S276), repeating or continuing ultrasound mediated delivery that is based on pressure-mediated, rather than temperature-mediated, effects.

15. The method of claim 14, said feedback being derived from in vitro diagnostic tests performed, respectively, before and after (S276, S290), said delivery delivers a therapeutic agent.

16. The method of claim 15, at least one of the tests being at least one of a a) blood-based assay (S278), b) urine-based assay (S286), and c) to measure molecular expression, a test utilizing a bodily substance other than blood or urine (S288).

17. A wearable device comprising:
an ultrasound mediated delivery module (315) for applying ultrasound to particles to deliver, as a payload, a therapeutic agent; and
a unit (320) configured for, automatically and without need for user intervention, both sampling body fluid and analyzing the samples.

18. The device of claim 17, said device configured for, automatically and without need for user intervention, regulating (380) the delivering responsive to a result of the analysis.

19. A method for ultrasound mediated delivery for which quantitative feedback is derived, comprising, in either order:
producing (S240) a transient pore through which to, by said delivery, deliver a therapeutic agent; and
attaching electrodes to, following said producing and in performing the deriving, take in vivo measurement of an electrical parameter responsive to permeability.

20. The method of claim 19, said attaching (S226) temporally following said producing.

21. A device configured for ultrasound mediated delivery; for applying energy to produce a transient pore through which to perform said delivery; and, for, after the pore is produced, administering said agent for said delivery through said pore (S252).

22. The device of claim 21, further configured for detecting that a site to which said applying is directed is clear (S250) of injected bubbles and for deferring said administering until after said detecting detects that said site is clear.

23. A computer readable medium for a substance delivery device, said medium comprising instructions executable by a processor for carrying out a series of acts, among which are the acts of, automatically and without need for user intervention: a) performing ultrasound mediated delivery so as to deliver a therapeutic agent via pressure-mediated, rather than temperature-mediated, effects (S234); b) deriving (S264), in real time, quantitative feedback; and c) proceeding based on the derived feedback.
